(19) United States
(12) Patent Application Publication
(10) Pub. No.: US 2009/0105580 A1
(43) Pub. Date: Apr. 23, 2009

(54) CHOOSING VARIABLES IN TISSUE VELOCITY IMAGING

(76) Inventor: Eirik Nestaas, Borgheim (NO)

Correspondence Address: KNOBBE MARTENS OLSON & BEAR LLP, 2040 MAIN STREET, FOURTEENTH FLOOR, IRVINE, CA, 92614, US

(21) Appl. No.: 12/095,073

(22) PCT Filed: Nov. 29, 2006
(86) PCT No.: PCT/NO2006/000439

§ 371 Date: Oct. 10, 2008

Related U.S. Application Data
(60) Provisional application No. 60/740,539, filed on Nov. 29, 2005.

(51) Int. Cl. A61B 5/05; A61B 8/00

(52) U.S. Cl. 600/411; 600/450

(57) ABSTRACT

The invention provides methods and systems for reducing noise in myocardial tissue velocity imaging such as ultrasound Doppler imaging or MRI. By adjusting recording factors and choosing apparatus settings and/or image analysis parameters in a systematic and consistent way, the quality of e.g. strain or strain rate imaging can be drastically improved. The invention reduces the noise by choosing values of variables, which lead to reduced or minimal beat-to-beat (BBV) variations in the imaged quantity such as average velocity, strain, strain rate or displacement or time derivatives of these.

### FIELD OF THE INVENTION

The present invention relates to tissue velocity imaging, such as ultrasound tissue Doppler imaging or tagged magnetic resonance imaging. More specifically, the invention relates to choosing apparatus settings or analysis parameters used in the image analysis.

### BACKGROUND OF THE INVENTION

Tissue velocity imaging is used to measure the velocity of moving tissue, most often in the myocardium. Generally, tissue velocity imaging can be divided into echocardiographic methods, such as Tissue Doppler Imaging (TDI) using ultrasound (often referred to as colour tissue velocity imaging or c-TVI), and magnetic resonance imaging (MRI) methods, such as three-dimensional tagged MRI.

Tissue velocity images contain a large amount of data, namely a one-, two- or three-dimensional velocity for each position in the tissue. The recent years have seen an increasing interest in values derived from the tissue velocities, especially the strain rate, see e.g. Stoylen et al., Echocardiography 16(4) (1999), 321-9 or Heimdal et al., J Am Soc Echocardiogr 11 (1998), 1013-9. The strain rate is a measure of the rate of deformation and is equivalent to the spatial derivative of the velocity. A negative strain rate means that the tissue segment is becoming shorter (or thinner), whereas a positive strain rate means that the segment is becoming longer (or thicker). Other derived values are for example the strain or the displacement. When monitoring such tissue velocity derived values wherein values over small regions are averaged or synthesized, the settings of the apparatus as well as the adjustment of parameters in the analysis have a large impact on the measurements.

Visual interpretation of myocardial function based on tissue velocity imaging has therefore shown to be dependent on the operator's experience. A long experience as well as practice and flair is required to be able to choose settings and analysis parameter values that lead to images, curves, peak values etc. which can be interpreted and which reflects the true state of the myocardium.

Further, when the myocardium becomes smaller, so does the size of the myocardial segments to be analysed, and position, shape and size of the region of interest becomes even more crucial.

For these reasons, assessing myocardial function using tissue velocity imaging is not an objective or reproducible procedure, and is presently not reliable as a diagnostic tool for children, neonates and smaller animals.

### SUMMARY OF THE INVENTION

In the present analysis of strain rate images, the selection of analysis parameters is to a large degree desultory. No systematic optimisation of the variables has been carried out, and selecting values for e.g. region of interest width or strain length is presently based on what is known to work, rather than on what has shown to work best by producing less noise.

It is an object of the invention to provide a method for reducing noise in tissue velocity imaging by adjusting recording factors and choosing apparatus settings and/or image analysis parameters in a systematic and consistent way.

It is another object of the invention to provide systems and applications for a tissue velocity image recording system, which automatically choose values for apparatus settings and/or image analysis parameters for tissue velocity derived value imaging.

In the present description, the term “tissue velocity image” refers to image or image data recorded with any apparatus capable of assigning velocities to a spatial (one- two- or three dimensional) distribution of myocardial tissue regions. Also, the term “tissue velocity derived value” refers to a value derived from myocardial tissue velocity data, e.g. strain, strain rate, displacement, or mean tissue velocity, which can be presented in image format.

The inventor of the present invention has realised an optimisation method applicable to several types of variables within tissue velocity imaging. According to a first embodiment of the invention, a method comprising the steps of monitoring a beat-to-beat variation (BBV) of a tissue velocity derived value in recorded tissue velocity image series, and varying the variables towards minimising said BBV is provided.

The invention may be applied to optimise different types of variables relating to different parts of tissue velocity imaging:
Recording factors. Factors external to the recording system, which affect the quality of the recorded image. These include e.g. anatomical or biological factors in the subject (e.g. size of the acoustic window, skeleton density and mineral content, whether the subject is at rest/not crying) and the performance of the operator (e.g. positioning of probe etc.).
Apparatus Settings. Internal settings in the recording apparatus, e.g. sampling rate, detector type.
Analysis parameters. Parameters chosen in the data analysis carried out by analysis software in relation to or externally from the system. Typical parameters are e.g. relating to the size and shape of the region of interest.

These three parts are illustrated in FIG. 1. The term “variables” refers to one or more recording factors, apparatus settings or analysis parameters. Since variables from each part affect the quality of or noise in the resulting image/curve/numbers, it follows that no result is better than the worst of these parts. In some situations, the noise originates mainly in one of the parts, in which case it may be sufficient to use the invention for properly choosing values within this part. The application of the method of the first embodiment may differ for each of the three types of variables, and they are therefore implemented as three individual embodiments in the following.

Hence, a second embodiment of the invention provides a method for choosing values of one or more analysis parameters for analysing image data in myocardial tissue velocity imaging, the method comprising the steps of:
recording a series of tissue velocity images of a myocardial segment over two or more heart beats;
systematically varying values of a set of one or more analysis parameters related to a tissue velocity image and, for each set of analysis parameter values, calculating a tissue velocity derived value in the segment for the series of images;
for each set of analysis parameter values estimating a BBV of the calculated tissue velocity derived value in the series of images;
choosing values for the set of analysis parameters that lead to a minimum BBV in the tissue velocity derived value.

The one or more analysis parameters may comprise any variable parameter, factor or coefficient used in the analysis of tissue velocity imaging and which affects the applied tissue velocity derived value.

Also, a third embodiment of the invention provides a method for choosing values of one or more apparatus settings in myocardial tissue velocity imaging, the method comprising the steps of:
systematically varying values of a group of one or more settings of a tissue velocity imaging apparatus, and, for each group of values, recording a series of tissue Doppler images of a myocardial segment over two or more heart beats with the apparatus,
calculating a tissue velocity derived value in the segment for each series of images;
for each group of values, estimating a BBV of the tissue velocity derived value in the corresponding series of images;
choosing values for the group of settings that lead to a minimum BBV in the tissue velocity derived value.

The methods of the second and third embodiments are preferably automatically carried out by a recording system. However, the variation and choice of analysis parameters and apparatus settings may be carried out by the operator based on the BBV's provided by the system.

Further, a fourth embodiment of the invention provides a method for adjusting one or more recording factors in a myocardial tissue velocity imaging set-up, the method comprising the steps of:
varying a recording factor and recording a series of tissue Doppler images of a myocardial segment over two or more heart beats,
calculating a tissue velocity derived value in the segment for the series of images;
estimating a BBV of the tissue velocity derived value in the corresponding series of images;
adjusting the one or more recording factors towards minimising the BBV in the tissue velocity derived value.

The method of the fourth embodiments is preferably carried out by the operator in co-operation with the recording system. The operator performs the recording and adjusts recording factors in response to the estimated BBV′ from the system.

The method of the second embodiments does not exclude varying also apparatus settings and/or recording factors as dealt with under the third and fourth embodiments, so that each BBV may be calculated for images using different apparatus settings and recording factors. Thereby, optimisation of apparatus settings or recording factors may be carried out simultaneously with the choosing of analysis parameters. Similarly, the method for choosing apparatus settings does not exclude varying also analysis parameters and/or recording factors, and the method for adjusting recording factors does not exclude varying also analysis parameters and/or apparatus settings.

In the following, common features of the methods of all embodiments of the invention will be described.

As indicated previously, the calculated tissue velocity derived value is preferably one of the following: strain, strain rate, displacement, tissue velocity, and time derivatives of these. Present tissue velocity derived value results are very much affected by noise, and the present invention provides the advantage of reducing this noise.

The statement that the variables should be chosen or adjusted to lead to a minimum BBV is not intended to mean that an absolute minimum value must be identified and chosen. As will be understood from the following specification, the value leading to an apparent or substantial minimum BBV is within the scope of the invention. When systematically varying values of a variable, values at regular intervals may be selected, and the one leading to the lowest BBV may be chosen. Alternatively, the BBV may be inter- or extrapolated to a value of the variable that leads to a minimum BBV and which lies in between or adjacent to the selected values, followed by selection of the inter- or extrapolated value for the variable. Optionally, a statistical significance of the difference between the values selected in the systematic variation can be estimated and taken into account in determining a variable value leading to a minimum BBV.

The BBV can be calculated using various methods providing an estimate of the difference in the tissue velocity derived value between heartbeats, and any approach that provides a value reflecting the BBV may be acceptable. In a preferred implementation, the BBV is calculated as the area between the curve describing the tissue velocity derived value in a first myocardial cycle and the curve describing the average between the first cycle and the foregoing cycle, divided by the area under the curve for the averaged cycle. Other applicable methods for calculating the BBV may be identified or developed by the skilled person. The BBV variation may be calculated based on values from more than two heartbeats, e.g. by using mean values taken over three or more beats.

It is preferred that the step of choosing values of the variable analysis parameters or apparatus settings comprises the steps of:
choosing, for a first analysis parameter or apparatus setting, a value leading to a minimum BBV in the tissue velocity derived value; and
choosing, for any additional analysis parameter or apparatus setting, a value leading to a minimum BBV in the tissue velocity derived value under the constrain of previously chosen values of other analysis parameters or apparatus settings.

As these steps are preferably carried out by a computer program using an applicable mathematical algorithm for carrying out continuous optimisation procedure, the steps may be interweaved or mixed.

The methods preferably further comprise the step of applying the chosen analysis parameter/setting value(s) to form a series of tissue velocity derived value images. Such formed image series represent a much more objective basis for using tissue velocity imaging in as a diagnostic tool and allows for an objective comparison between image series taken under different conditions or on different subjects. Also, image series formed in this way may be the output from any apparatus or system applying the methods. The method for choosing analysis parameters is preferably carried out every time a new tissue velocity derived value image series is to be obtained. The method for choosing apparatus settings is preferably carried out every time a new subject or a new segment of a subject heart is to be examined. The method of adjusting recording factors may be applied by the operator continuously or when needed.

The methods also encompass choosing or adjusting any new or not mentioned values derived from or depending on the tissue velocity data. The steps of calculating the tissue velocity derived value and calculating or estimating the BBV may preferably be repeated for other tissue velocity derived values than the one applied. Thereby, BBVs of the other tissue velocity derived values may be calculated or estimated and taken into account when choosing analysis parameter/setting values.

As previously mentioned, the present invention may be applied in relation to different kinds of tissue velocity imaging.

In one embodiment, the invention relates to ultrasound tissue Doppler imaging. In this embodiment, the tissue velocity images are ultrasound tissue Doppler images, and the tissue velocity imaging apparatus or system is an ultrasound tissue Doppler imaging system.

Here, the one or more analysis parameters may comprise one or more of the following: strain length, region of interest length, region of interest width, region of interest shape, region of interest area, averaging techniques (time window, Gaussian/linear), drift compensation, etc.

In the choosing of settings in the ultrasound tissue Doppler imaging apparatus, the one or more apparatus settings may comprise one or more of the following: phase range, velocity range, wavelength, frequency, frame rate, spatial resolution, temporal resolution, type of probe, second harmonic techniques, lateral velocity averaging, depth velocity averaging, etc.

Also, the recording factors may comprise one or more of the following: size of acoustic window; the skills and experience of the operator translating into position, orientation, and movement of the ultrasound probe in relation to the subject; movement of the torso region of the subject—at rest/not crying (neonates/infants); respiration rate of the subject, pulse of the subject, the presence of reverberations and acoustic shadows, etc.

In another embodiment, the invention relates to MRI. In this embodiment, the tissue velocity images are three-dimensional tagged magnetic resonance images, and the tissue velocity imaging apparatus or system is a MRI apparatus capable of performing three-dimensional tagged MRI.

In this embodiment, similar or equivalent recording factors, apparatus settings, or analysis parameters may be applied, as well as other variables specific to MRI.

The use of the present invention within both Ultrasound and MR imaging applies equally to the following embodiments.

As all of the recording factors, apparatus settings, and analysis parameters may be chosen or adjusted according to the present invention, a fifth embodiment of the invention provides a method for improving recording and analysis in myocardial tissue velocity imaging, the method comprising adjusting recording factors for the recording of tissue velocity images using the method of the fourth embodiment, choosing settings for the tissue velocity imaging system using the method of the third embodiment, and choosing analysis parameters for the analysis of recorded images using the method of the second embodiment.

Today's tissue velocity imaging apparatuses or systems are complicated machinery controlled by complex electronic processing systems having a user interface for controlling recording and analysis of images. Therefore, the various aspects of the present invention may be implemented in an electronic processing system controlling tissue velocity imaging apparatus, e.g. as software and/or hardware components.

Hence, a sixth embodiment of the invention provides a tissue velocity imaging system implementing the method of the second embodiment. The tissue velocity imaging system preferably has an image analysis component for analysing recorded tissue velocity image data and presenting it to a user, the image analysis component comprising:
means for accessing recorded tissue velocity image data;
an application for choosing values of a set of one or more analysis parameters used in generating tissue velocity images of a myocardial segment, the application comprising:
   means for systematically varying values of the set and calculating a tissue velocity derived value in the segment for each set of values;
   means for calculating/estimating a beat-to-beat variation (BBV) of the tissue velocity derived value in a series of tissue velocity images for each set of values;
   means for choosing values for the set of analysis parameters that lead to a minimum BBV in the tissue velocity derived value;
means for generating tissue velocity images using analysis parameter value(s) chosen by the application; and
a graphical interface for presenting generated tissue velocity images to the user.

In this context, a component is one of the individual parts of which a control system of the imaging system is made up. The control section may include both hardware, software, and interfaces for both the user and the remaining sections of the system. Also, an application is a program that gives a computer instruction to provide the user with tools to accomplish a task.

The means comprised by the application for choosing values are preferably all software means, such as parts of a computer program.

A seventh embodiment of the invention provides a software application corresponding to the application for choosing values of the sixth embodiment. The software application may be a computer program or a part of a computer program, which may be loaded into the memory of a control system for a tissue velocity imaging system and executed there from. The computer program may be distributed by means of any data storage or data transmission medium, e.g. the Internet. The storage media may be e.g. CD-ROM, mini-disc, hard disk, ferro-electric/magnetic memory, flash memory, read only memory (ROM), random access memory (RAM), USB memory keys, etc.

An eighth embodiment of the invention provides a tissue velocity imaging system implementing the method of the third embodiment. The tissue velocity imaging system preferably has a component for setting apparatus settings, the component comprising:
means for accessing recorded tissue velocity image data;
an application for choosing values of a set of one or more apparatus settings in myocardial tissue velocity imaging, the application comprising:
   means for systematically varying values of a group of one or more settings of a tissue velocity imaging apparatus, and, for each group of values, recording a series of tissue Doppler images of a myocardial segment over two or more heart beats with the apparatus,
   means for calculating a tissue velocity derived value in the segment for each series of images;
   means for, for each group of values, estimating a beat-to-beat variation (BBV) of the tissue velocity derived value in the corresponding series of images;
   means for choosing values for the group of settings that lead to a minimum BBV in the tissue velocity derived value.
means for setting apparatus settings chosen by the application.

If good quality recordings in a population (e.g. neonates, premature infants, adults) is carried out using optimised apparatus settings and analysis parameters, it will be possible to estimate the normal or expected BBV of a population (population specific normal values) in good quality images. Using the same group of apparatus settings during image recording and using the same set of parameters during analysis, the BBV estimate might be used for evaluation of the recording factors, i.e. the quality of the image-recording situation. Hence the estimated BBV from the method of adjusting recording factors (fourth embodiment) might be helpful when evaluating the uncertainness/quality component of the recording situation, by quantifying the noise component.

Thereby, this BBV may also help the operator in optimising the recording situation, or may be used to train or guide the operator in performing recordings. For this purpose, a ninth embodiment of the invention provides a tissue velocity imaging system implementing the method of the fourth embodiment. The tissue velocity imaging system preferably has a recording guide component for guiding an operator in adjusting recording factors in the recording of tissue velocity images of a myocardial segment, the recording guide component comprising:
means for accessing recorded tissue velocity image data;
an application for calculating a real-time quality estimate of the recorded images, the application comprising:
   means for calculating a tissue velocity derived value of the myocardial segment for recorded images;
   means for continuously estimating a beat-to-beat variation (BBV) of the tissue velocity derived value;
   means for deriving a quality estimate based on the estimated BBV; and
      a graphical interface for continuously presenting the quality estimate to the operator.

The system of the ninth embodiment thereby provides a real-time feedback to the operator, relating to the quality of the recording situation. This will allow the operator to practice his/her skills and can thereby be used as a practice or educational system. Additionally, it may function as a guide in difficult or abnormal recording situations, where recording factors which cannot be varied (e.g. subject anatomy) make optimal recording difficult.

Strain and strain rate imaging are noisy methods, and the noise level often exceeds the strength of the signals originating in the movement of the biological tissue. The basic idea of the invention is to reduce the noise in tissue velocity images by adjusting recording factors, apparatus settings, and/or analysis parameters under the assumption that succeeding heartbeats are equivalent for subjects in rest. This is generally a good assumption, as data from other imaging techniques indicate that the biological BBV is several times smaller than the variations which can be attributed to noise. Under this assumption, the inventor realised that choosing recording factors, apparatus settings, and/or analysis parameters that lead to minimum BBV's of one or more tissue velocity derived values is an excellent tool for noise reduction in tissue velocity imaging.

### BRIEF DESCRIPTION OF THE FIGURES

Embodiments of the invention will be described, by way of example only, with reference to the drawings, in which

FIG. 1 illustrates the different parts in tissue velocity imaging.

FIGS. 2A-C are graphs illustrating the calculation of the BBV of a tissue velocity derived value.

FIG. 3 is a graph with a curve illustrating the BBV of the strain rate, f_{SR}[v(t)], under variation of the ROI length, L_{ROI}.

FIG. 4 is a drawing illustrating the different analysis parameters in tissue velocity imaging.

FIGS. 5 through 10 are graphs illustrating strain and strain rate BBVs as a function of different analysis parameters.

FIGS. 11A and B are graphs illustrating strain and strain rate BBVs as a function of different apparatus settings.

FIG. 12 shows a general layout of a tissue velocity imaging system according to the invention.

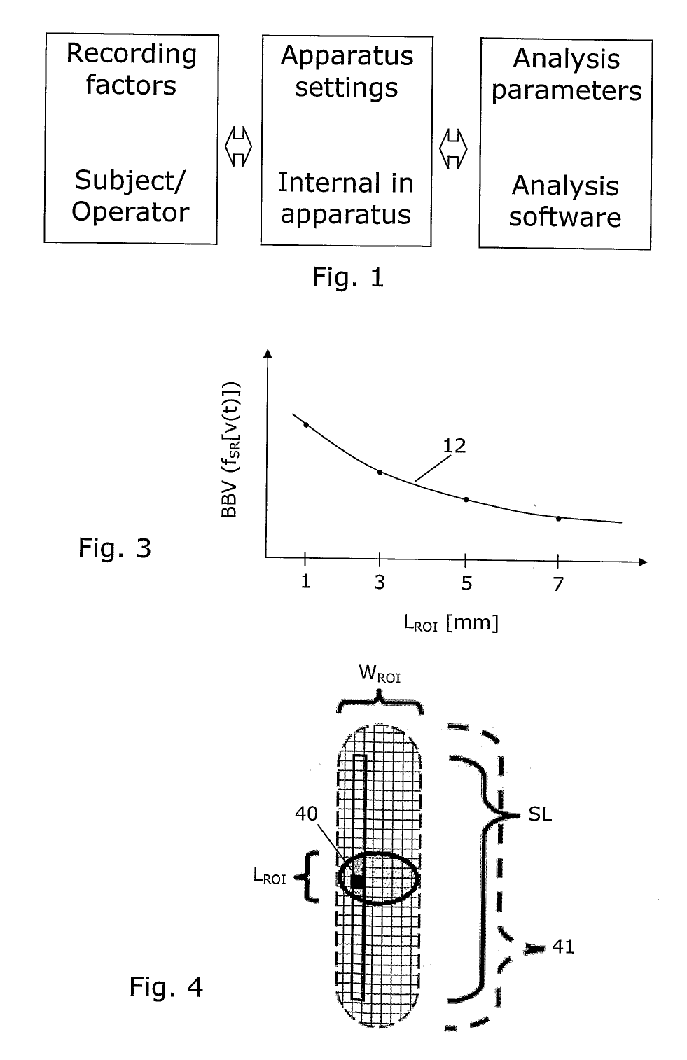

### DETAILED DESCRIPTION OF THE INVENTION

The detailed description will disclose and enable embodiments of the invention using examples within ultrasound tissue velocity imaging. The equivalent applications of the embodiments within other tissue velocity imaging techniques will be within the realms of the skilled person.

First, a general outline of the methods according to the embodiments will be given. Thereafter a detailed example will be given using experimental data and data analysis. Finally, embodiments of the system and software implementations of the invention will be presented.

The following abbreviations will be used throughout the description:

TABLE 1

| Term/concept | Abbreviation |
|---|---|
| Beat-to-beat variation | BBV |
| Region of interest | ROI |
| Region of interest length | L_{ROI} |
| Region of interest width | W_{ROI} |
| Strain length | SL |
| Time or number of image within a series | t |
| Tissue velocity derived value | f[v(t)] |
| Mean Velocity | f_{MV}[v(t)] |
| Displacement | f_{D}[v(t)] |
| Strain Rate | f_{SR}[v(t)] |
| Strain | f_{S}[v(t)] |
| Number of parameter/setting permutations | N |
| Counter for sets of parameter/setting values | n |
| Analysis parameter value | P |
| Apparatus setting value | S |
| Recording factor value | F |
| Counters for parameters/settings | m, i |
| Two-dimensional myocardial tissue velocity | 2D-MTVI |
| image | |

The process of recording a myocardial tissue velocity image can be divided into three parts, each involving variables contributing to the quality of or noise in the final image, curve or value;
Recording factors, e.g. an acoustical window of the apparatus, position of an ultrasound probe in relation to a subject, orientation of an ultrasound probe in relation to a subject, movement of an ultrasound probe in relation to a subject, movement of the torso region of a subject, respiration rate of a subject, pulse of a subject, etc.
Apparatus settings, e.g. phase range, velocity range, wavelength, frequency, frame rate, spatial resolution, temporal resolution, type of probe, second harmonic techniques, lateral velocity averaging, depth velocity averaging, etc.
Analysis parameters, e.g. strain length, region of interest length, region of interest width, region of interest shape, region of interest area, averaging techniques (time window, Gaussian/linear), drift compensation, etc.

Different sets of variables will give rise to different amounts of noise in the final result (images, curves, values). There is not one set of variables that gives a minimum noise level, as the noise level in different images of a series can be different for at given set of variables, and as the noise level in different regions can be different for at given image.

The various methods for choosing variable values differ in some steps, mainly relating to when and by whom/what they are carried out. Tables 2, 3, and 4 sum up the method steps to be carried out in the choosing of analysis parameters and apparatus settings, and the adjustment of recording factors. The different steps will be described in greater detail below.

TABLE 2
Choosing analysis parameters

| 1 | Record one series of images over at least two heartbeats | |
|---|---|---|
| 2 | Select analysis parameters (Pₘ) to be varied | |
| 3 | Systematically vary parameter values, N sets of permutations | |
| 4 | For each set of | i. calculate fₙ[v(t)] for the series of images |
| | parameter values, n | ii. calculate BBV of fₙ[v(t)] over the series |
| | | of images |
| 5 | Determine BBVₙ as a function of a first setting, S₁ | |
| 6 | Select value of S₁ corresponding to Min\|BBVₙ\| | |
| 7 | For any additional setting | determine BBVₙ as a function of setting |
| | S_{m>1} | Sₘ using only BBVₙ's with selected values |
| | | of S_{i<m} |
| | | select value of Sₘ corresponding to |
| | | Min\|BBVₙ\| |

TABLE 3
Choosing apparatus settings

| A | Select apparatus settings (Sₘ) to be varied | |
|---|---|---|
| B | Systematically vary settings, N sets of permutations | |
| C | For each set of settings, | i. record a series of images over at least |
| | n (each image series) | two heartbeats. |
| | | ii. calculate fₙ[v(t)] for the series of |
| | | images |
| | | iii. calculate BBV of fₙ[v(t)] over the |
| | | series of images |
| D | Determine BBVₙ as a function of a first setting, S₁ | |
| E | Select value of S₁ corresponding to Min\|BBVₙ\| | |
| F | For any additional | determine BBVₙ as a function of setting |
| | setting S_{m>1} | Sₘ using only BBVₙ's with selected |
| | | values of S_{i<m} |
| | | select value of Sₘ corresponding to |
| | | Min\|BBVₙ\| |

TABLE 4
Adjusting recording factors

| | |
|---|---|
| I | Select recording factor (F) to be varied |
| II | Select a new value of F and, while holding the value of F constant, |
| | record a series of images over at least two heartbeats |
| III | Calculate f[v(t)] for the series of images |
| IV | Determine the BBV of f[v(t)] |
| V | Repeat steps II-IV a total of two or more times |
| VI | Adjust the value of F to the value with Min\|BBV\| |
| VII | Select another recording factor (F) to be varied and repeat steps I-VI |

Steps 4(i)/C(ii)/III. The tissue velocity derived values are calculated for each image or, equivalently, for each time step according to the temporal resolution of the recorded image series. The calculation formulas depend on the desired tissue velocity derived values, some typical (generalised) formulas are given here, and others exist.

Average velocity within ROI:

$$\overline{v} ( t ) = \frac{{\sum }_{q} {v}_{q} ( t )}{n} ,$$

where q is the number of velocity values in the ROI.

Strain rate:

$$S R = \frac{v ( r ) - v ( r + Δ r )}{Δ r} ,$$

where r is the position in the ROI:

Displacement of ROI:

$$D ( t ) = {\int }_{{t}_{0}}^{t} \overline{v} ( t ) d t$$

Strain in ROI:

$$ɛ ( t ) = \frac{D ( t ) - D ( {t}_{0} )}{D ( {t}_{0} )}$$

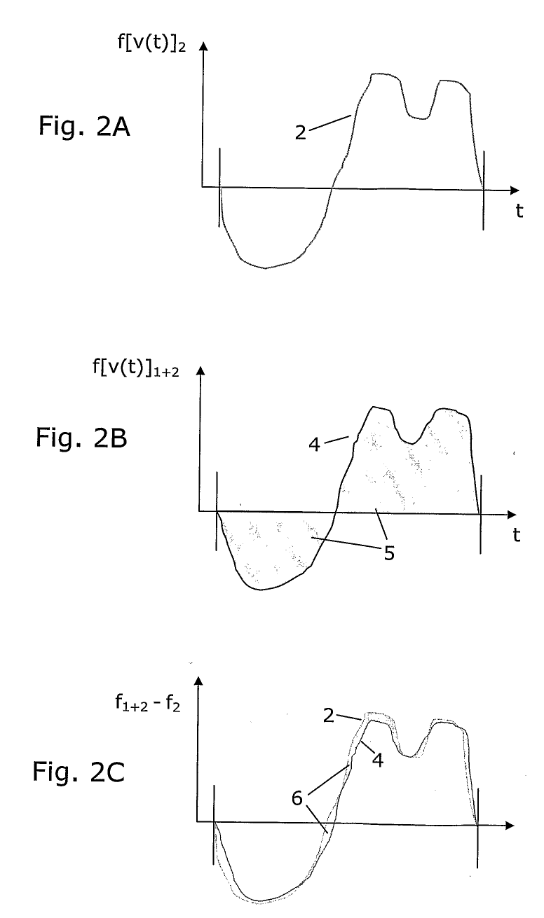

Steps 4(ii)/C(iii)/IV. The BBV can be calculated using various methods for providing an estimate of the difference in the tissue velocity derived value between two heartbeats. In the detailed example to be presented later, the BBV is calculated as the area between the curve describing the tissue velocity derived value in a first cardial cycle and the curve describing the average between the first cycle and the foregoing cycle, divided by the area under the curve for the averaged cycle. Referring to FIG. 2A, curve 2 shows a tissue velocity derived value calculated over cardial cycle 2. Similarly, in FIG. 2B, curve 4 shows the average curve of the tissue velocity derived value calculated over myocardial cycles 1 and 2. To estimate the difference, curves 2 and 4 are subtracted to obtain the areas 6 between them. This is shown in FIG. 2C. The total sum of areas 6 are then divided by accumulated area 5 under curve 4, and the resulting scalar is the BBV. This method of calculating the BBV was selected primarily because it was easy to extract these data using the applied data analysis software. Numerous of other methods for estimating the BBV can be applied.

Steps 5/D. Now, having obtained BBVs for each set of analysis parameter/setting values, the dependency of the BBV on each analysis parameter/setting can be determined. FIG. 3 shows a graph with an example curve 12 illustrating the BBV of the strain rate (f_{SR}[v(t)]) under variation of the ROI length L_{ROI}.

Steps 6/E. As can be seen from the curve 12, the BBV decreases for increasing ROI lengths. Increasing the ROI length even further may lead to a lower BBV, as values will be averaged over a larger region. But increasing the ROI length beyond the size of the monitored myocardial segment will not provide valuable data, so it is not of interest to increase the ROI length beyond 7 mm in two-segmental analysis of neonates (in adults, ROI lengths of up to 30 mm has been used). Hence, selecting analysis parameter values for the systematic variation plays an important role in choosing the analysis parameter value leading to minimum BVV (here choosing the optimal ROI length)—only analysis parameter values which are applicable and which provide valuable output should be included in the permutations.

Steps 7/F. In the example of FIG. 2, if ROI length was the first analysis parameter to be chosen, one should choose L_{ROI}=7 mm as this leads to the minimum BBV. However, if the ROI length was an additional analysis parameter, it may be subject to constraints from previously chosen analysis parameter values, e.g. chosen strain length together with requirements that SL+L_{ROI}≦9 mm, or chosen ROI width together with a fixed ROI area.

### DETAILED EXAMPLES

In the following, a study applying the invention to optimise analysis parameters in a real recording scenario is presented. Later, a study applying the invention to optimise apparatus settings is presented.

Global left ventricle systolic function is obtained in neonates by parameters like shortening fraction and ejection fraction. Strain and strain rate can be used to assess such regional myocardial function. The aim of the study is to find a valid and reliable way to measure strain and strain rate in healthy term neonates. The influence of different SL, ROI lengths and ROI widths on the measured BBV in strain and strain rate is studied, and then the best combination of ROI size and SL is found which allows for a two-segment analysis in term neonates.

When strain and strain rate are estimated from two-dimensional myocardial tissue velocity images (2D-MTVI), the deformation for each point can be calculated using the velocity gradient along a line centred at that point and parallel with the ultrasound beam, the strain length. The regional strain and strain rate are studied within a ROI, as illustrated in FIG. 4. The strain and strain rate for each point 40 within the ROI are estimated by using the velocities along each points strain length and the regional values are averaged from these points. The sum of the strain length and the ROI length defines the length of the area from which the velocities for the regional deformation analysis are collected and should therefore not exceed the length of the segment 41. The relative weight of the velocities within the segment is determined by the ROI length to strain length ratio. Choosing equal strain length and ROI length, the velocities within the centre of the segment is weighted more than the velocities towards the ends of the segment, while if either the strain length or the ROI length is larger than the other the velocities are weighted more evenly.

With a short strain length the velocity gradient estimate is less accurate because the gradient is estimated from fewer velocities and the velocity differences are smaller. With a small ROI the regional value is averaged from fewer velocity gradients. Small ROIs and short strain lengths will therefore resulting in a more unfavourable signal-to-noise ratio. Adult hearts are larger than neonatal hearts (ventricle length 10 cm vs. 3 cm, wall thickness 6-10 mm vs. 3-4 mm), supposed to lead to a less favourable signal-to-noise ratio in neonates than in adults.

The measured BBV between two consecutive heart cycles is caused by the true beat to beat variation and the noise component. The deformation estimations are noisy methods and the measured strain and strain rate BBV would therefore mainly be caused by the noise component. Between two consecutive heart cycles the true beat to beat variation is small. A small measured BBV between two consecutive heart cycles would therefore reflect a small noise component in the deformation analysis, and the noise components for the different combinations of ROI sizes and strain lengths can be compared by their BBVs.

### Materials

Forty-eight term neonates were included in the project and were investigated during the first, the second and the third day of life. Five apical projections were used to study nine walls. Ten good-quality 2D MTVI images from each wall were included in this study. The left lateral wall, the septum and the right lateral wall were studied from the 4-chamber view. From the left 2-chamber view the left inferior and left superior walls were studied, and from the long axis view the left anterior and left posterior wall were studied. Two additional apical projections of the right ventricle were used. From the 4-chamber view the probe was tilted to get the right ventricle in centre. By rotating the probe clockwise the right superior free wall was studied, and by rotating the probe contraclockwise the right inferior free wall was studied. The tissue velocity datasets were recorded with the wall parallel to the ultrasound beams using tissue velocity range −16 to 16 cm/sec, transducer frequency 2424 kHz and pulse frequency 1000 kHz (5S probe, Vivid 7, GE Vingmed, Horten, Norway).

### Ultrasound Analyses

Using analysis software (Echopac PC SW 4.0.x, GE Vingmed, Horten, Norway), two segments were investigated in each wall, using linear drift compensation for the Lagrangian strain curves, 40 ms Gaussian smoothing and elliptic shaped ROIs. The BBV was defined as described previously in relation to FIGS. 2A-C.

To study the effect of different SLs, ROI length and ROI widths on the strain and strain rate BBV, the apical and basal segment in each wall were investigated using ROI lengths of 1, 3 and 6 mm, ROI widths of 1, 2, 3 and 4 mm, and strain lengths of 4, 6, 8 and 10 mm. The strain and strain rate BBV were estimated for each of these 48 combinations. In each segment all ROIs were equally centred and traced using a semiautomatic tracking system to compensate for the myocardial movement during the cardiac cycle.

The end-systolic length of the myocardial walls parallel to the ultrasound beam were 2.5 cm or higher in this study. To allow two-segment analyses in each wall without interference from adjacent segments, we therefore regarded segment size 9-12 mm as appropriate and sought the lowest strain and strain rate BBV in combinations with the sum of the ROI length and the strain length (L_{ROI}+SL) within this range.

### Statistics

The One way ANOVA and posthoc Scheffe test was used to differ between the BBV for the different settings. Regression analyses were used to compare the impact of increased ROI length on the BBV at different SLs, and multiple regression analyses were used to adjust for the effect of changing ROI area when comparing the effect of different ROI widths on the BBV. When searching for the optimal settings, we the used One way ANOVA and Scheffe post hoc test to excluded combinations statistically significantly different from the best found, and then repeated the procedure until no statistically significant differences was found between the remaining combinations. Two sided p-values and 95% confidence intervals were used. To determine the inter- and intra observer variation we used the strain and strain rate BBV interclass correlations for one randomly selected 2D MTVI from each of the walls, investigated twice by the same operator several weeks apart.

### Influence of the ROI Length, Width and Strain Length on the BBV

Both the strain BBV and the strain rate BBV differed significantly between the different SLs (Table 5) and also between the different ROI lengths (Table 6) (One way ANOVA, post hoc Scheffe test, p<0.05 for all pair wise comparisons). The strain BBV and strain rate BBV were both statistically significantly influenced by the ROI lengths at each SL, and the SLs at each ROI length (One way ANOVA, p<0.05 for both analyses).

TABLE 5
Impact of strain length on the BBV. Mean and 95% confidence interval

| Strain length (mm) | Strain BBV | Strain rate BBV |
|---|---|---|
| 4 | 0.1365 (0.1318-0.1412)¹ | 0.2560 (0.2511-0.2608)² |
| 6 | 0.1155 (0.1118-1192)¹ | 0.2274 (0.2233-0.2315)² |
| 8 | 0.0880 (0.0853-0.0906)¹ | 0.1910 (0.1876-0.1944)² |
| 10 | 0.0799 (0.0775-0.0822)¹ | 0.1782 (0.1750-0.1814)² |

¹Statistically significantly different from the strain BBV at the other strain lengths (p < 0.05)
²Statistically significantly different from the strain rate BBV at the other strain lengths (p < 0.05)

TABLE 6
Impact of ROI length on the BBV. Mean and 95% confidence interval.

| ROI length (mm) | Strain BBV | Strain rate BBV |
|---|---|---|
| 1 | 0.1201 (0.1165-0.1238)¹ | 0.2336 (0.2296-0.2376)² |
| 3 | 0.1060 (0.1030-0.1091)¹ | 0.2156 (0.2121-0.2192)² |
| 6 | 0.0877 (0.0863-0.0911)¹ | 0.1902 (0.1872-0.1933)² |

¹Statistically significantly different from the strain BBV at the other ROI lengths (p < 0.05)
²Statistically significantly different from the strain rate BBV at the other ROI lengths (p < 0.05)

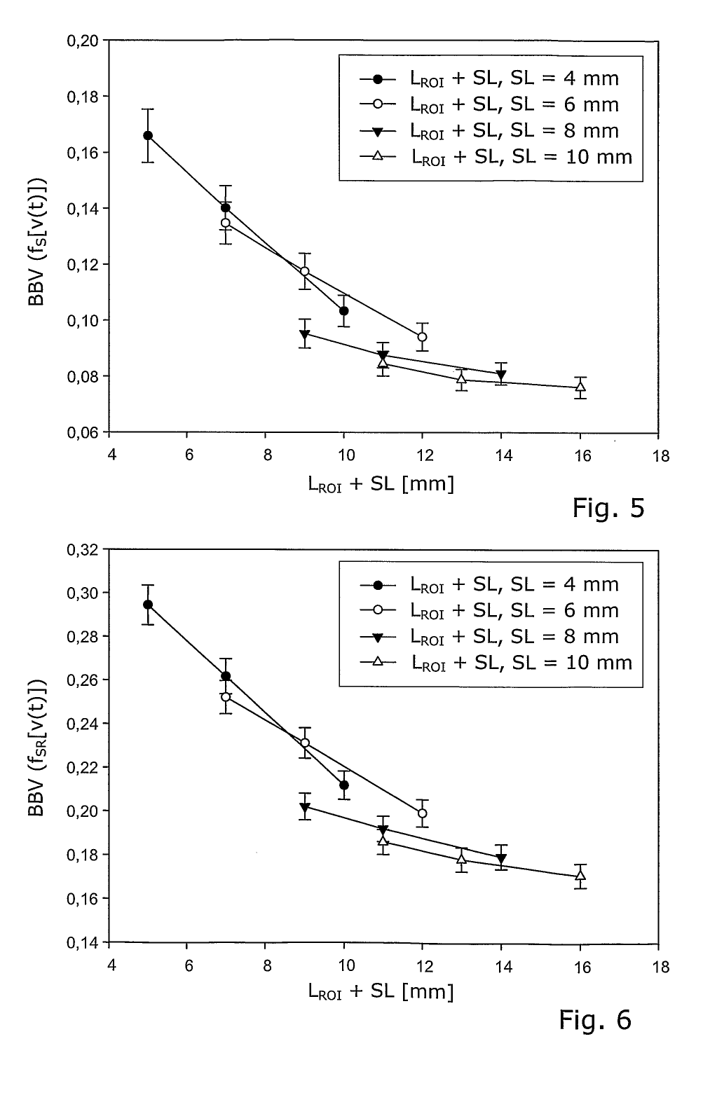

FIGS. 5 and 6 shows the impact of different combinations of ROI length (L_{ROI}) and strain length (SL) on the strain (FIG. 5) and strain rate (FIG. 6) BBV, dots and bars indicates mean and 95% confidence interval. As can be seen, the changes in BBV between the different ROI lengths were most pronounced at the shortest SLs.

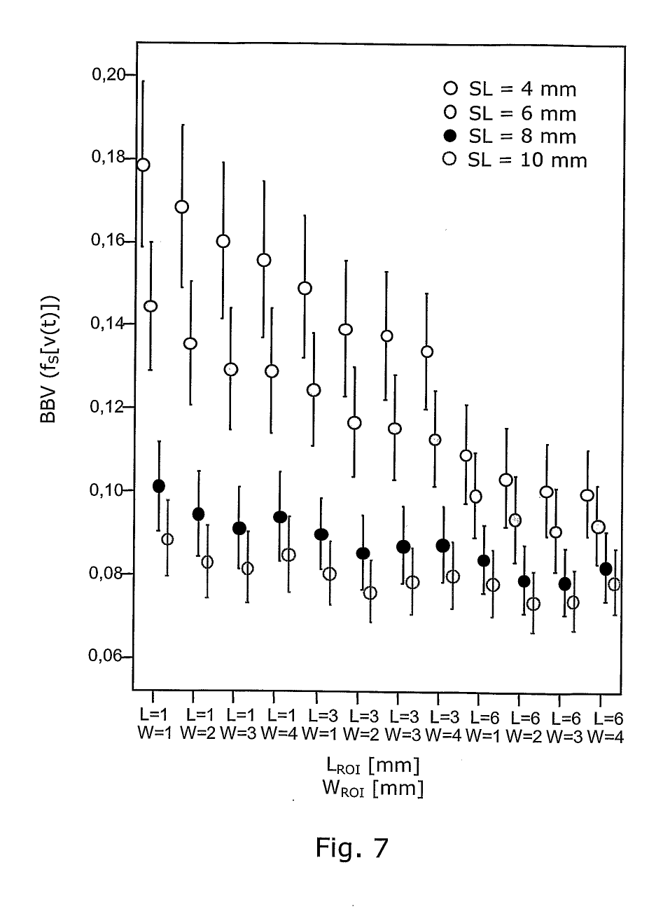

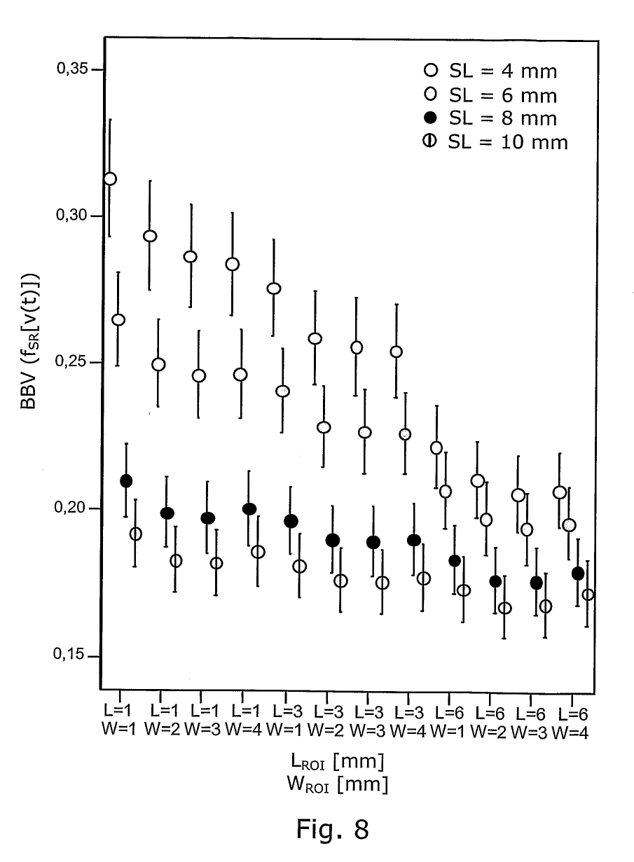

A similar data analysis was made for the ROI shape and area, by investigating the impact of different combinations of ROI length (L_{ROI}) and ROI width (W_{ROI})) on the strain and strain rate BBV. The results are summarised in FIGS. 7 (strain) and 8 (strain rate), dots and bars indicates mean and 95% confidence interval.

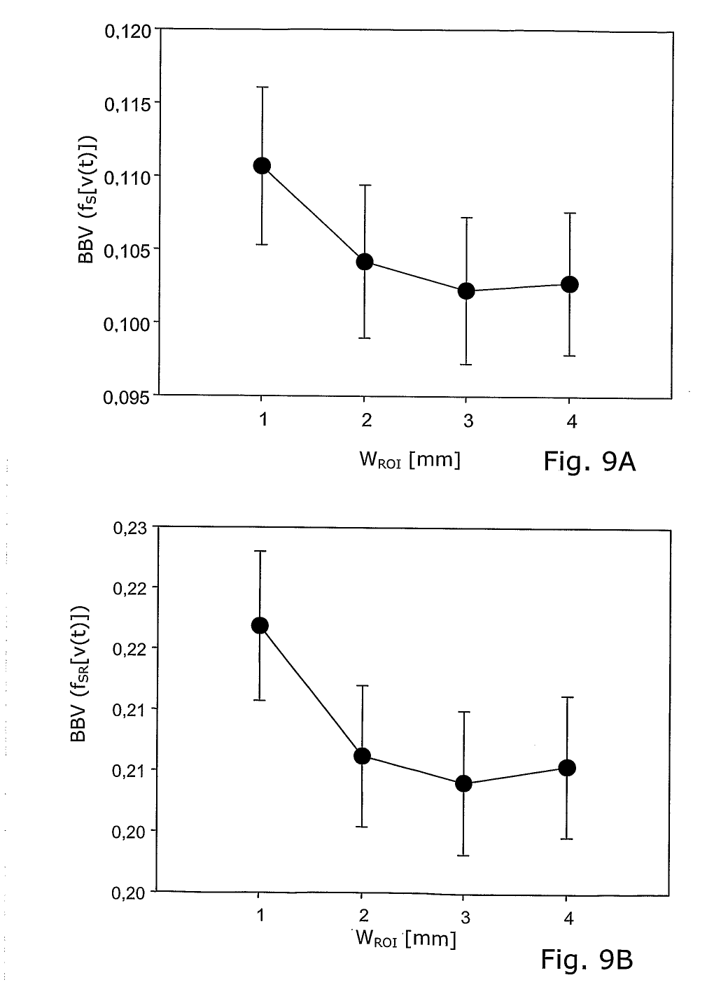

FIGS. 9A and B show the strain and strain rate BBV as a function of ROI widths. The strain BBV (FIG. 9A) at ROI width 1 mm is not statistically significantly different from the strain BBV at 2 mm, but is statistically significantly different from the strain BBV at ROI width 3 mm and at 4 mm. The strain rate BBV (FIG. 9B) at ROI width 1 mm is statistically different from the BBV at the other ROI widths. There is no statistically significant difference between the strain BBV or strain rate BBV at 2, 3 and 4 mm ROI widths, neither when all ROI widths are compared nor when ROI width 1 mm is excluded (One way ANOVA, post hoc Scheffe test).

To adjust for the changing ROI area when studying the effect of different ROI widths on the strain and strain rate BBVs, multiple regression analyses were performed for the dependence on ROI area and ROI width. A positive ROI width regression factor represents a decreased quality per point at increased ROI widths. In the basal segments there were statistically significant influence from the ROI width both on the strain BBV (B=0.007, p<0.05) and strain rate BBV (B=0.008, p<0.05). In the apical segments, there was no statistically significant influence from ROI width on the strain BBV (p>0.05), while the statistically significant influence on the strain rate BBV (B=0.004, p<0.05) was smaller than in the basal segments. From this, it can be seen that noise in strain/strain rate increases by increasing ROI widths, an analysis made possible by using analysis parameters chosen using the present invention.

### Optimizing Analysis Parameters for Neonates

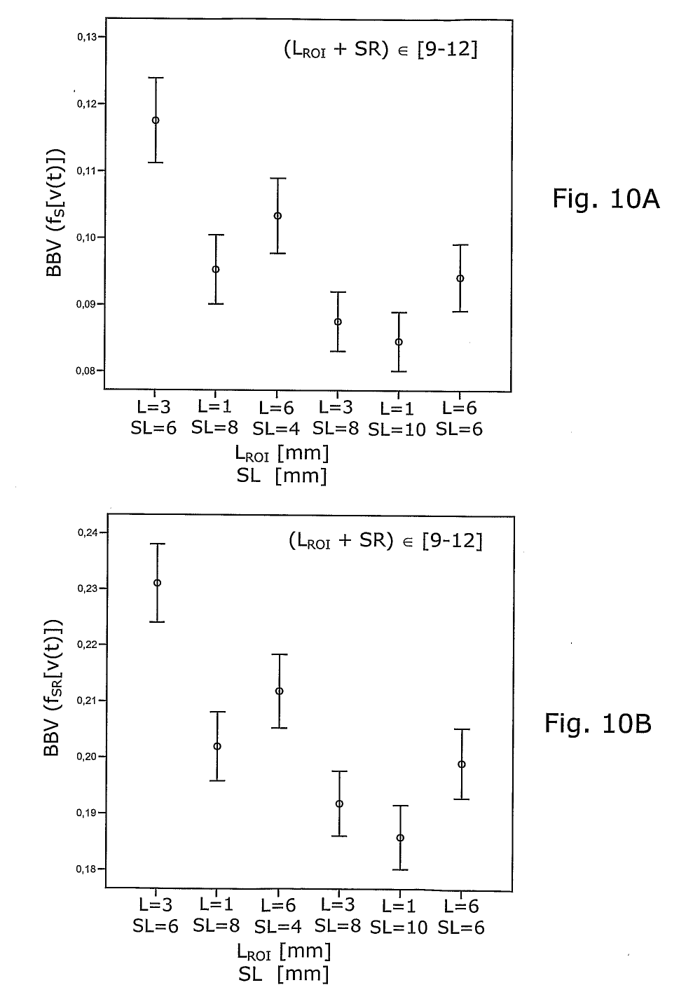

When searching for an optimized combination of ROI size and SL for use in term neonates, all combinations with sum of ROI length and SL within the range 9-12 mm were compared. FIGS. 10A and B show the strain (10A) and strain rate (10B) BBV for these six combinations, dots and bars indicate mean and 95% confidence interval. Of these six combinations, both the lowest strain BBV and strain rate BBV was found in the combination of ROI length 1 mm and strain length 10 mm.

When comparing this combination towards the other combinations and excluding statistically significantly different combinations stepwise (one way ANOVA, post hoc Scheffe test, p<0.05), both the strain BBV and strain rate BBV were statistically significantly higher in all others except the combination of ROI length 3 mm and SL 8 mm.

For the combination of ROI length and 1 mm strain length 10 mm, there were no statistically significant differences in the strain BBVs or the strain rate BBVs between the different ROI widths (one way ANOVA p>0.05). Both the strain BBV and the strain rate BBV were lowest at ROI width 3 mm, the strain BBV was 0.0817 (0.0731-0.903) (mean and 95% confidence interval) and the strain rate BBV was 0.1823 (0.1710-0.1935).

### Intra- and Inter-Observer Results

One randomly selected 2D-MTVI from each of the eight walls were analysed twice by the same operator (EN) several weeks apart. The intra observer strain BBV interclass correlation was 0.58 and the intra observer strain rate BBV interclass correlation was 0.72. The lowest strain BBV and strain rate BBV were in both cases found using the combination of SL 10 mm, ROI length 1 mm and ROI width 3 mm.

### Influence of L_{ROI}, W_{ROI}, and SL on the BBV.

When analysing longitudinal strain and strain rate in short segments, the strain length should be kept long on the expense of ROI length to reduce the BBV. When using a long strain length, the BBV of the velocity gradient is reduced because the velocity gradient is estimated from a larger number of velocities and because the velocity differences are greater. This reduces the BBV of the estimated deformation for each point within the ROI. Increasing the ROI length will increase the ROI area and hence the number of points from which the regional deformation is calculated. The effect of increased ROI lengths on the BBVs was smaller than the effect of increased strain length, especially at long strain lengths. When increasing the ROI width, the benefit of the increased ROI area was countered by the higher noise (lower quality of the signal) in the new points. In our data, these effects balanced both for strain and strain rate at ROI width 2-4 mm. Both the strain BBV and the strain rate BBV formed a “U”-shaped curve when plotted against the ROI width, and the BBVs were lowest using ROI width 3 mm. However, neither the strain nor the strain rate BBV at ROI width 3 mm were statistically significantly different from the BBVs at ROI widths 2 mm or 4 mm.

### Optimizing Analysis Parameters for Neonates

When comparing combinations suitable for two-segment analyses in term neonates, both the lowest strain BBV and the lowest strain rate BBV were found using ROI length 1 mm and strain length 10 mm. When using these combinations, both the strain BBV and the strain rate BBV were lowest using ROI width 3 mm. However, there were no statistically significant differences at this combination of strain length and ROI length between the different ROI widths, and there were also no statistically significant differences between the combination of ROI length 3 mm and strain length 8 mm and the combination of ROI length 1 mm and strain length 10 mm.

### Notes on the Experiment

The difference in deformation estimates between using the combination of a long strain length and a short ROI length and the combination of a short strain length and a long ROI length has not been studied. In both cases, the sum of the strain length and the ROI length defines the length of the segment from which the tissue velocities are collected. The relative weight of the velocities within the segments depend on the chosen ROI length and strain length, and velocity differences unevenly distributed within the segment might therefore have different impact on the regional deformation estimates in the different combinations.

When searching for the optimal combination of strain length and ROI size in neonates, the combinations with BBVs statistically different from the lowest were excluded and the procedure then repeated until no statistically significant differences were found between the remaining combinations. By choosing this approach, some of the combinations were compared more than once. To compensate for the multiple testing, a conservative statistical test (Scheffe test) was chosen for the pair wise comparisons. The interclass correlations for the intra observer variation were not very high. However, the lowest BBVs within each of the intra-observer observation were found using the same combination of ROI size and strain length, both for the strain BBV and the strain rate BBV.

### Optimizing Apparatus Settings and Analysis Parameters

The following describes measurements applying embodiments of the invention to select apparatus settings (probe type and frame rate) as well as analysis parameters (W_{ROI}, L_{ROI}, SL) during TVI recording and deformation analysis in term neonates.

The strain and SR beat to beat variation were assessed in 8 good-quality TVI for each of the following probe and frame rate (FR) settings (Vivid 7, GE Vingmed, range +/−16 cm/sec);
5S probe default FR (FR_{d})
10S default FR (FR_{d})
10S low FR (FRₗ)

The 10S probe (default ultrasound frequency 8.0 MHz, pulse frequency 2000 Hz) is mainly used in premature and term newborns. The 5S probe has default ultrasound frequency 2.4 MHz, and pulse frequency 1000 Hz. When performing the recordings the frame rate and beam density is related. Increasing the frame rate will reduce the beam density and then the accuracy for each velocity measurement will decrease, but if time-based smoothing is used, each reported value will be averaged from more velocities. The noise in the recordings might differ between probes. Low frequency probes penetrate the tissue more deeply than high frequency probes. High frequency probes often provide more detailed information (higher spatial resolution) within the area that the beams can reach. It is not known whether a high frame rate or a high beam density will provide the best signal to noise ratio. Further, it is not known whether the optimal settings during the off-line analyses (strain and strain rate analyses) are similar for the different settings during the tissue velocity recordings.

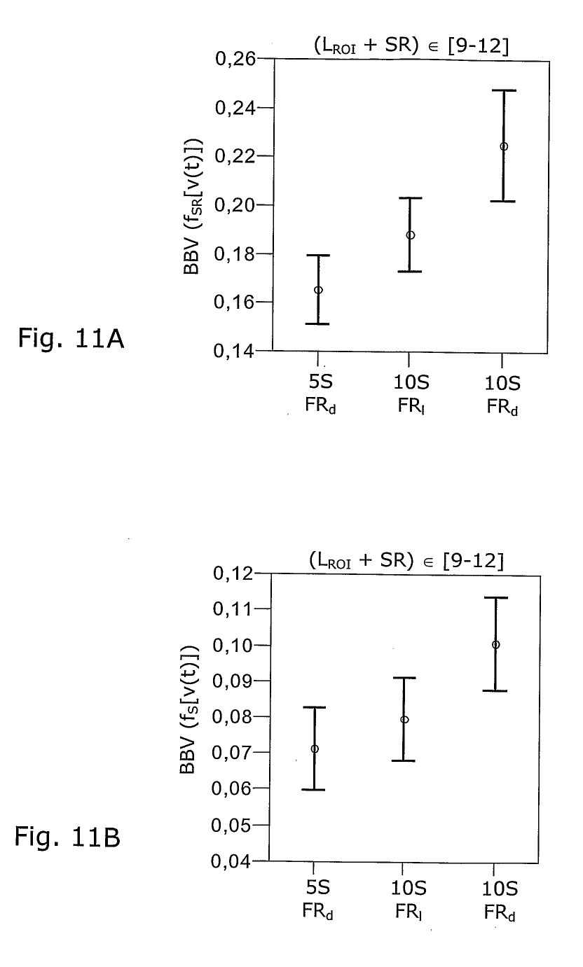

Two segments per wall were analysed using 48 different combinations of ROI size and SL. FIGS. 11A and B illustrate the BBV of the strain length and the strain for the different probe and frame rate combinations. The bars indicate the 95% confidence interval of the noise component for the different combinations of ROI size and SL in the analysis. As can be seen, both BBVs were lower in the 5S than in both the 10S series (p<0.05), indicating less noise in the 5S probe.

Table 7 shows the analysis parameters leading to the smallest strain and strain rate BBV for the different settings.

TABLE 7

| Series | Optimal SL | Optimal L_{ROI} | Optimal W_{ROI} |
|---|---|---|---|
| 5S defalt FR | 10 mm | 1 mm | 2 mm £ |
| 10S low FR * | 10 mm | 1 mm | 1 mm $ |
| 10S high FR *¤ | 10 mm | 1 mm | 1 mm # |

* Strain rate BBV significantly higher than the 5S series
¤ Strain BBV significantly higher than the 5S and the 10S low framerate series
£ Significant higher strain BBV at ROI width 1 mm.
$ Significant differences between ROI width 1, 2, 3 and 4 mm for both BBVs
# Significant difference between ROI width 1, 2, 3 and 4 mm for the strain BBV, the difference for the strain rate BBV did not reach significance (p = 0.054)

As can be seen from Table 7, both BBVs decreased with increased SL in each series (p<0.05). Except for the 10S default FR strain BBV (p=0.086), both BBVs decreased with increased ROI length (p<0.05). Of the combination of ROI length and SL eligible for two-segment analyses, the lowest BBVs in all series were found using ROI length 1 mm and SL 10 mm. The optimal ROI width was smaller using the 10S probe (1 mm) than the 5S probe (3 mm).

Thus, the BBVs can be used to assess the optimal settings and parameters during TVI recording and analysis. The BBVs were lower using the 5S probe than the 10S probe. In two-segment analysis, the optimal ROI length was 1 mm and SL was 10 mm, and the optimal ROI width was 1 mm using the 10S probe and 3 mm using the 5S probe.

Although the above examples applied the methods of choosing analysis parameters and apparatus settings, it is within the realms of the skilled person to carry out similar processes using the methods for adjusting recording factors according to the invention.

### System and Software

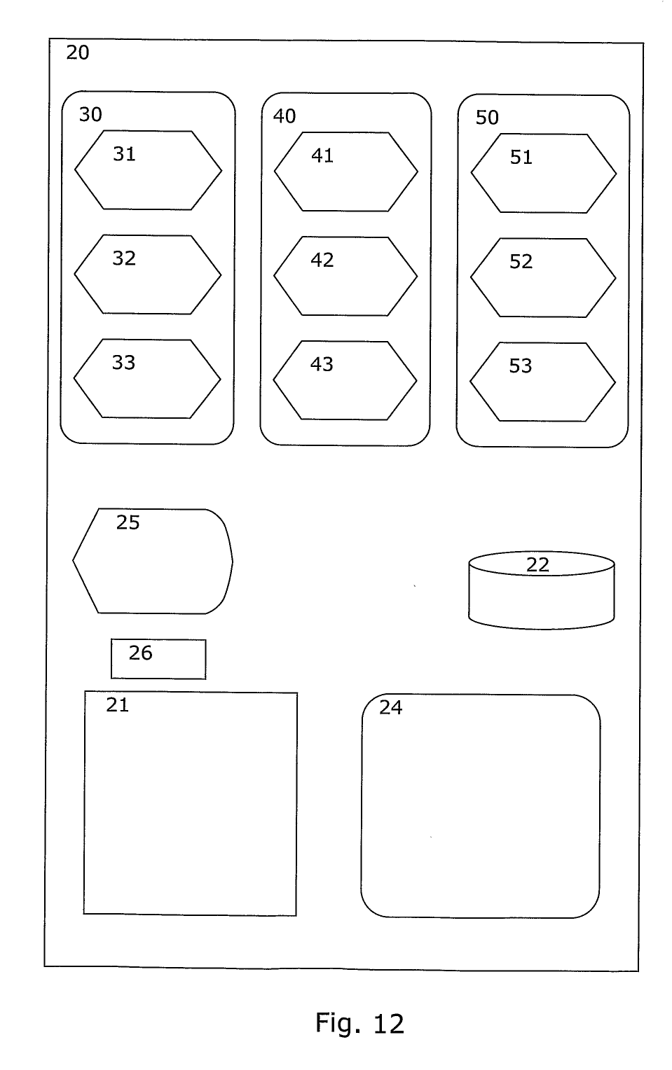

FIG. 12 shows a layout of a tissue velocity imaging system 20 with an image analysis component 30 for choosing values of analysis parameters according to one embodiment, a component 40 for setting apparatus settings according to another embodiment, and/or a recording guide component 50 according to yet another embodiment of the invention. The system has a section 21 for recording images and a data storage 22 for storing recorded image data. Control of recording processes and handling of data is carried out by an electronic processor system 24, user interface is carried out through display 25 and input 26, e.g. keyboard or a mouse and a GUI.

The image analysis component 30 also comprises means 31 for accessing recorded tissue velocity image data as well as means 32 for generating tissue velocity images using chosen parameter values. The means 31 and 32 are typically standard functions in existing velocity imaging software, where the user has specified the desired analysis parameter values. The image analysis component 30 also has an application 33 for choosing values for analysis parameters according to the method described in relation to Table 2. The application 33 can be software designed to analyse the recorded tissue velocity image data and choose analysis parameters which is then fed to the means 32 so that tissue velocity images are generated using these values. The application 33 thereby performs the function of the experienced user, in that it specifies the parameter values to be used. The application 33 can be integrated in the standard velocity imaging software, or it can be executed as a separate applet simply sending the determined analysis parameters to the means 32.

The component 40 for setting apparatus settings also comprises means 41 for accessing recorded tissue velocity image data as well as means 42 for setting the chosen apparatus settings. The means 41 and 42 are typically standard functions in existing velocity imaging software, since most apparatus settings are controlled via a computer interface. However, in case the apparatus setting encompasses the probe type as in the example described previously, the means 42 for setting the apparatus settings could be the operator physically changing the probe. The component 40 for setting apparatus settings also has an application 43 for choosing values for apparatus settings according to the method described in relation to Table 3. The application 43 for choosing values for apparatus settings can be a computer program which either interfaces with the apparatus to change settings, or which provides the operator with the changes in the settings to be performed.

The recording guide component 50 comprises means 51 for accessing recorded tissue velocity image data as well as a graphical interface 52 for continuously presenting the quality estimate to the operator. The recording guide component 50 also has an application 53 for instructing the operator or patient to use a given recording factor, and calculate a real-time quality estimate, the result of which may be shown on display 25. The application 53 for can be a computer program designed according to the method described in relation to Table 4.

The application 53 will can guide the operator to make recordings with reduced noise by continuously giving feedback on the BVV or quality estimate of the recording. This also offers the possibility of using the tissue velocity imaging system 20 to train personnel on how to make recordings with low noise.

In the above description, certain specific details of disclosed embodiments such as specific factors, settings, parameters, designs etc, are set forth for purposes of explanation rather than limitation, so as to provide a clear and thorough understanding of the present invention. However, it should be understood readily by those skilled in this art, that the present invention might be practiced in other embodiments which do not conform exactly to the details set forth herein, without departing significantly from the spirit and scope of this disclosure. Further, in this context, and for the purposes of brevity and clarity, detailed descriptions of well-known analysis processes, apparatus, methodology, etc. have been omitted so as to avoid unnecessary detail and possible confusion.

1. A method for optimising variables in tissue velocity imaging, the method comprising: monitoring a beat-to-beat variation (BBV) of a tissue velocity derived value in recorded tissue velocity image series, and varying the variables towards minimising said BBV.

2. A method for choosing values of one or more analysis parameters for analysing image data in myocardial tissue velocity imaging, the method comprising:
recording a series of tissue velocity images of a myocardial segment over two or more heart beats;
systematically varying values of a set of one or more analysis parameters related to a tissue velocity image and, for each set of analysis parameter values, calculating a tissue velocity derived value in the segment for the series of images;
for each set of analysis parameter values, estimating a beat-to-beat variation (BBV) of the calculated tissue velocity derived value in the series of images; and
choosing values for the set of analysis parameters that lead to a minimum BBV in the tissue velocity derived value.

3. The method according to claim 2, further comprising the step of applying the chosen analysis parameter value(s) to form a series of tissue velocity derived value images.

4. The method according to claim 2, wherein the tissue velocity images are ultrasound tissue Doppler images.

5. The method according to claim 2, wherein the tissue velocity images are three-dimensional tagged magnetic resonance images.

6. The method according to claim 2, wherein the one or more analysis parameters comprise one or more of the following: strain length, region of interest length, region of interest width, region of interest shape, region of interest area, time window or Gaussian/linear averaging techniques, or drift compensation.

7. A method for choosing values of one or more apparatus settings in myocardial tissue velocity imaging, the method comprising:
systematically varying values of a group of one or more settings of an tissue velocity imaging apparatus, and, for each group of values, recording a series of tissue Doppler images of a myocardial segment over two or more heart beats with the apparatus;
calculating a tissue velocity derived value in the segment for each series of images;
for each group of values, estimating a beat-to-beat variation (BBV) of the tissue velocity derived value in the corresponding series of images; and
choosing values for the group of settings that lead to a minimum BBV in the tissue velocity derived value.

8. The method according to claim 7, further comprising the step of recording a series of tissue velocity images using the chosen setting value(s).

9. The method according to claim 7, wherein the tissue velocity imaging apparatus is an ultrasound tissue Doppler imaging apparatus.

10. The method according to claim 9, wherein the one or more settings comprise one or more of the following: phase range, velocity range, wavelength, frequency, frame rate, spatial resolution, temporal resolution, type of probe, second harmonic techniques, lateral velocity averaging, or depth velocity averaging.

11. The method according to claim 7, wherein the tissue velocity imaging apparatus is a magnetic resonance imaging (MRI) apparatus capable of performing three-dimensional tagged MRI.

12. The method according to claim 2, wherein the step of choosing values comprises:
choosing, for a first analysis parameter in the set or setting in the group, a value leading to a minimum BBV in the tissue velocity derived value; and
choosing, for any additional analysis parameter in the set or setting in the group, a value leading to a minimum BBV in the tissue velocity derived value under the constrain of previously chosen values of other analysis parameters or settings.

13. The method according to claim 2, wherein the steps of calculating the tissue velocity derived value and calculating the BBV are repeated for another tissue velocity derived value, and wherein the BBVs of the other tissue velocity derived value are taken into account when choosing analysis parameter values.

14. A method for adjusting one or more recording factors in a myocardial tissue velocity imaging set-up, the method comprising:
varying a recording factor and recording a series of tissue Doppler images of a myocardial segment over two or more heart beats;
calculating a tissue velocity derived value in the segment for the series of images;
estimating a beat-to-beat variation (BBV) of the tissue velocity derived value in the corresponding series of images;
adjusting the one or more recording factors towards minimising the BBV in the tissue velocity derived value.

15. The method according to claim 14, wherein the tissue velocity imaging is performed using an ultrasound system, and wherein the one or more recording factors comprise one or more of the following: an acoustical window of the apparatus, position of an ultrasound probe in relation to subject, orientation of an ultrasound probe in relation to subject, movement of an ultrasound probe in relation to subject, movement of the torso region of a subject, respiration rate of a subject, or pulse of a subject.

16. The method according to claim 1, wherein the calculated tissue velocity derived value is one of the following: strain, strain rate, displacement, tissue velocity and time derivatives of these.

17. A method for improving recording and analysis in myocardial tissue velocity imaging, the method comprising adjusting recording factors for the recording of tissue velocity images using the method according to claim 14, choosing settings for the tissue velocity imaging system using the method according to claim 7 and choosing analysis parameters for the analysis of recorded images using the method according to claim 2.

18. A tissue velocity imaging system comprising an image analysis component for analysing recorded tissue velocity image data and presenting it to a user, the image analysis component comprising:
a means for accessing recorded tissue velocity image data;
an application for choosing values for a set of one or more analysis parameters used in generating tissue velocity images of a myocardial segment, the application comprising:
a means for systematically varying values of the set and calculating a tissue velocity derived value in the segment for each set of values;
a means calculating or estimating a beat-to-beat variation (BBV) of the tissue velocity derived value in a series of tissue velocity images for each set of values;
a means for choosing values for the set of analysis parameters that lead to a minimum BBV in the tissue velocity derived value;
a means for generating tissue velocity images using analysis parameter value(s) chosen by the application; and
a graphical interface for presenting generated tissue velocity images to the user.

19. A software application for choosing values of a set of one or more analysis parameters used in generating tissue velocity images of a myocardial segment, the application comprising:
a means for systematically varying values of the set and calculating a tissue velocity derived value in the segment for each set of values;
a means for calculating or estimating a beat-to-beat variation (BBV) of the tissue velocity derived value in a series of tissue velocity images for each set of values; and
a means for choosing values for the set of analysis parameters that lead to a minimum BBV in the tissue velocity derived value;

20. A tissue velocity imaging system comprising a component for setting apparatus settings, the component comprising:
a means for accessing recorded tissue velocity image data;
an application for choosing values for a group of one or more apparatus settings in myocardial tissue velocity imaging, the application comprising:
a means for systematically varying values of a group of one or more settings of a tissue velocity imaging apparatus, and, for each group of values, recording a series of tissue Doppler images of a myocardial segment over two or more heart beats with the apparatus;
a means for calculating a tissue velocity derived value in the segment for each series of images;
a means for, for each group of values, estimating a beat-to-beat variation (BBV) of the tissue velocity derived value in the corresponding series of images;
a means for choosing values for the group of settings that lead to a minimum BBV in the tissue velocity derived value; and
a means for setting apparatus settings chosen by the application.

21. A tissue velocity imaging system comprising a recording guide component for guiding an operator in adjustment of recording factors in the recording of tissue velocity images of a myocardial segment, the recording guide component comprising:
a means for accessing recorded tissue velocity image data;
a an application for calculating a real-time quality estimate of the recorded images, the application comprising:
a means for calculating a tissue velocity derived value of the myocardial segment for recorded images;
a means for continuously estimating a beat-to-beat variation (BBV) of the tissue velocity derived value;
a means for deriving a quality estimate based on the estimated BBV; and
a graphical interface for continuously presenting the quality estimate to the operator.
